(19) United States
(12) Patent Application Publication
(10) Pub. No.: US 2012/0317874 A1
(43) Pub. Date: Dec. 20, 2012

(54) Nitrogen Free Deposit Control Fuel Additives

(75) Inventors: Paul R. Stevenson, Belper (GB); David J. Moreton, Milford (GB); Jonathan S. Vilardo, Chardon, OH (US); Seth Crawley, Mentor, OH (US); James C. Ray, Mentor, OH (US); Kurt F. Wollenberg, Chardon, OH (US)

(73) Assignee: THE LUBRIZOL CORPORATION, Wickliffe, OH (US)

(21) Appl. No.: 13/084,656

(22) Filed: Jun. 15, 2011

(51) Int. Cl. C10L 1/234; C07C 43/205; C10L 1/19; C07C 43/164; C10L 1/185; C10L 1/188

(52) U.S. Cl. 44/351; 44/447; 44/385; 44/437; 44/388; 44/403; 568/662

(57) ABSTRACT

The present invention provides a nitrogen-free fuel detergent additive, fuel additive compositions containing the same, and fuel compositions containing the same, for use in internal combustion engines, where the additive is the reaction product of (i) hydrocarbyl phenol, cresol or similar material and (ii) a aldehyde, in the presence of an optional catalyst, wherein the additive provides acceptable engine deposit control that is comparable and/or better than the deposit control provided by nitrogen-containing fuel additives.

### BACKGROUND OF THE INVENTION

The present invention relates to fuel additives, fuel additive compositions and fuel compositions as well as a method for fueling an internal combustion engine, providing improved deposit control inside the engine, as well as other benefits, with additives that are free of nitrogen.

Hydrocarbon based fuels generally contain numerous deposit-forming substances. When used in internal combustion engines (ICE), deposits from these substances can form on and around constricted areas of the engine which come in contact with the fuel. In these ICE, such as automobile engines, deposits can build on engine intake valves leading to progressive restriction of the gaseous fuel mixture flow into the combustion chamber, in turn reducing the maximum power of the engine, decreasing fuel economy, increasing engine emissions, and hindering engine startability.

As engines have and continue to become more sensitive to deposits, due to engine designs including tighter clearances with more constructed areas and other reasons, it is common practice to incorporate a detergent into the fuel composition used in the engine for the purpose of reducing or inhibiting the formation of, and facilitating the removal of, engine deposits. These additives improve the engine performance and reduce the engine emissions.

Generally, fuel detergent additives include additives that can be described as ashless dispersants. These additives consist of hydrocarbyl backbones, including polyisobutylene (PIB) backbones, that have been combined with polar, nitrogen-containing head groups. The primary fuel detergent additives used today include PIB amines, PIB succinimides and PIB phenol Mannich amines. One key aspect of these fuel detergent additives is the presence of an active nitrogen-containing group, which is believed to be required for good performance of the additives.

In some cases, nitrogen-containing additives can lead to undesirable effects, such as seal degradation, particularly in the case of elastomer containing seals. Nitrogen-free additives would be free of these potential disadvantages.

There is a need for an effective fuel additive that may be used in fuel additive compositions and fuel compositions and in the operation of internal combustion engines that is free of nitrogen. There is need for such nitrogen-free additives that provide comparable and/or improved performance compared to the nitrogen-containing additives commonly used today.

### SUMMARY OF THE INVENTION

A new class of fuel detergents have been discovered which offer improvements over traditional fuel detergents such as polyisobutylene (PIB) phenol Mannich detergents. This new class of detergents does not contain any nitrogen, traditionally believed to be essential to the good performance of fuel detergent additives while still delivering comparable and/or improved performance compared to the nitrogen-containing additives commonly used today.

The present invention provides a composition comprising a nitrogen-free detergent fuel additive represented by Formula I;

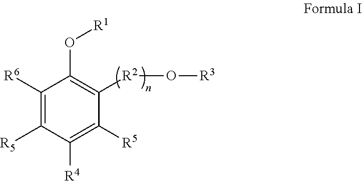

wherein: R¹ is independently hydrogen or a hydrocarbyl group containing 1 to 50 carbon atoms; R² is independently a hydrocarbyl group containing 1 to 10 carbon atoms; n is 0 or 1; R³ is independently hydrogen or a hydrocarbyl group containing 1 to 50 carbon atoms; R⁴ is a hydrocarbyl group containing 1 to 150 carbon atoms; each R⁵ is independently hydrogen or a hydroxyl group; R⁶ is independently hydrogen or a hydrocarbyl group containing 1 to 50 carbon atoms or —(R⁷)ₘ—OR⁸ wherein R⁷ is a hydrocarbyl group containing 1 to 10 carbon atoms, R⁸ is hydrogen or a hydrocarbyl group containing 1 to 50 carbon atoms and m is 0 or 1; and wherein R¹, R², R³, R⁴, R⁵, R⁶, R⁷ and R⁸ are free of nitrogen.

In some embodiments the additives of the present invention are represented by Formula II:

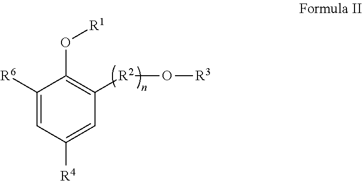

wherein: R¹ is hydrogen or a hydrocarbyl group containing 1 to 6 carbon atoms; R² is a hydrocarbyl group containing 1 to 6 carbon atoms; R³ is hydrogen or a hydrocarbyl group containing 1 to 6 carbon atoms; R⁴ is a polyisobutylene group having a number average molecular weight of 350 to 5000; and R⁶ is independently hydrogen or a hydrocarbyl group containing 1 to 50 carbon atoms or —(R⁷)ₘ—OR⁸ wherein R⁷ is a hydrocarbyl group containing 1 to 10 carbon atoms, R⁸ is hydrogen or a hydrocarbyl group containing 1 to 50 carbon atoms and m is 0 or 1. In some embodiments the R⁶ in Formula I and/or Formula II is independently hydrogen or a hydrocarbyl group containing 1 to 50 carbon atoms

The various definitions of each of the R groups listed above for Formula II may also be applied to Formula I shown above.

In some embodiments the additive of the present invention comprises a mixture of one or more additives from the following list: a) the additive represented by Formula II wherein R³ is hydrogen and R⁶ is hydrogen; b) the additive represented by Formula II wherein R³ is a hydrocarbyl group containing 1 to 6 carbon atoms and R⁶ is hydrogen; c) the additive represented by Formula II wherein R³ is hydrogen and R⁶ is a hydrocarbyl group containing 1 to 6 carbon atoms; d) the additive represented by Formula II wherein R³ is a hydrocarbyl group containing 1 to 6 carbon atoms and R⁶ is a hydrocarbyl group containing 1 to 6 carbon atoms

In other embodiments the additive of the present invention comprises a mixture of one or more additives from the following list: a) the additive represented by Formula I wherein R³ is hydrogen, each R⁵ is hydrogen and R⁶ is hydrogen; b) the additive represented by Formula I wherein R³ is a hydrocarbyl group containing 1 to 6 carbon atoms, each R⁵ is hydrogen and R⁶ is hydrogen; c) the additive represented by Formula I wherein R³ is hydrogen, each R⁵ is hydrogen and R⁶ is a hydrocarbyl group containing 1 to 6 carbon atoms; d) the additive represented by Formula I wherein R³ is a hydrocarbyl group containing 1 to 6 carbon atoms, each R⁵ is hydrogen and R⁶ is a hydrocarbyl group containing 1 to 6 carbon atoms.

In still further embodiments, the additives of the present invention are represented by either Formula I and/or Formula II above wherein R¹ is hydrogen; R² is independently a methylene group, an ethylene group, a propylene group or a butylene group; R³ is independently hydrogen, a methyl group, an ethyl group, a propyl group or a butyl group; R⁵ is hydrogen; R⁶ is independently hydrogen, a methyl group, an ethyl group, a propyl group or a butyl group; or combinations thereof. In some embodiments, in combination with one or more of the embodiments described above, R² is a methylene group, R³ is a methyl group and R⁶ is a hydrogen group, hydroxy group or a methyl group.

The present invention also provides for a fuel additive composition and/or concentrate comprising: one or more of the nitrogen-free detergent additives described herein; an optional solvent; and one or more optional additional performance additives.

The present invention also provides for a fuel composition comprising: one or more of the nitrogen-free detergent additives described herein; a fuel; and one or more optional additional performance additives.

The present invention also provides for a method of operating an internal combustion engine, comprising supplying to said engine a fuel composition comprising one or more of the nitrogen-free detergent additives described herein; a fuel; and one or more optional additional performance additives.

The present invention also provides for a process for making the nitrogen-free detergent fuel additives of the present invention comprising reacting a hydrocarbyl-substituted hydroxy aromatic compound and an aldehyde wherein the reaction is optionally carried out in the presence of a catalyst.

### DETAILED DESCRIPTION OF THE INVENTION

Various preferred features and embodiments will be described below by way of non-limiting illustration.

### Field of the Invention

The present invention involves a fuel additive, a fuel additive composition, a fuel composition and a method for fueling an internal combustion engine, where the fuel additive is free of nitrogen.

The fuel additive composition of the invention shows comparable and/or improved engine deposit control, allowing for improved engine performance, including but not limited to reductions in deposit-caused engine power losses, reduction in deposit-caused fuel economy losses and decreases in deposit-caused engine emissions. The fuel detergent additive may also be used as a corrosion inhibitor or a lubricity aid.

### The Nitrogen Free Additive

The nitrogen-free fuel detergent additives of the present invention have been shown to effectively control the formation of engine deposits, including intake valve deposits. This result is unexpected as it is generally believed that a nitrogen-containing polar group is required for a fuel additive to provide effective deposit control in an engine. While not wishing to be bound by theory, the polar nitrogen-containing group is believed to be necessary for good performance as the polar head may effectively associate with dirt and/or deposit particles in an engine, allowing them to be dispersed by the fuel additive and facilitate for their removal from, and/or prevent their initial deposit on, engine surfaces. With no polar group present, it is believed the additive would be much less effective at associating with dirt and deposit particles in the engine, and so would be much less effective at controlling engine deposits.

The present invention provides nitrogen-free fuel detergent additives that are effective at controlling engine deposits, despite the fact that they are free of nitrogen, and so free of any polar nitrogen-containing groups.

While not wishing to be bound by theory, it is believed that the nitrogen-free fuel detergent additives provide effective deposit control due, at least in part, to the proximity of the ortho-polar group in the adjacent position to the phenolic (or cresol) hydroxyl group or ether, as illustrated in Formula I, and Formula II, shown above.

The fuel detergent additive of the present invention is represented by Formula I, shown above, wherein: R¹ is independently hydrogen or a hydrocarbyl group containing 1 to 50, 1 to 25, 1 to 10, or 1 to 6 carbon atoms; R² is independently a hydrocarbyl group containing 1 to 10, or 1 to 6 carbon atoms; n is 0 or 1; R³ is independently hydrogen or a hydrocarbyl group containing 1 to 50, 1 to 25, 1 to 10, or 1 to 6 carbon atoms; R⁴ is a hydrocarbyl group containing 1 to 150, 10 to 150, or 50 to 150 carbon atoms; each R⁵ is independently hydrogen or a hydroxyl group; R⁶ is independently hydrogen or a hydrocarbyl group containing 1 to 50 carbon atoms or —(R⁷)ₘ—OR⁸ wherein R7 is a hydrocarbyl group containing 1 to 10 carbon atoms, R8 is hydrogen or a hydrocarbyl group containing 1 to 50 carbon atoms and m is 0 or 1; and wherein R¹, R², R³, R⁴, R⁵, R⁶, R⁷ and R⁸ are free of nitrogen.

The hydrocarbyl groups that make up the various groups defined above are not particularly limited. Suitable hydrocarbyl groups include polyolefins prepared by polymerizing olefin monomers by well known polymerization methods that are also commercially available. Suitable olefin monomers include monoolefins, including monoolefins having 2 to 10 carbon atoms such as ethylene, propylene, 1-butene, isobutylene, and 1-decene. An especially useful monoolefin source is a C₄ refinery stream having a 35 to 75 weight percent butene content and a 30 to 60 weight percent isobutene content. Useful olefin monomers also include diolefins such as isoprene and 1,3-butadiene. Olefin monomers can also include mixtures of two or more monoolefins, of two or more diolefins, or of one or more monoolefins and one or more diolefins. Useful polyolefins include polyisobutylenes having a number average molecular weight of 140 to 5000, in another instance of 400 to 2500, and in a further instance of 140 or 500 to 1500 or 1100. The polyisobutylene can have a vinylidene double bond content of 5 to 69%, in a second instance of 50 to 69%, and in a third instance of 50 to 95%. The polyolefin can be a homopolymer prepared from a single olefin monomer or a copolymer prepared from a mixture of two or more olefin monomers. Also possible as the hydrocarbyl substituent source are mixtures of two or more homopolymers, two or more copolymers, or one or more homopolymers and one or more copolymers.

The hydrocarbyl-substituted phenol can be prepared by alkylating phenol with an olefin or polyolefin described above, such as a polyisobutylene or polypropylene, using well-known alkylation methods.

In one embodiment, the vinylidene content of the R⁴ hydrocarbyl group in Formula I and/or Formula II can comprise at least about 30 mole % vinylidene groups, at least about 50 mole % vinylidene groups, or at least about 70 mole % vinylidene groups. Such material and methods for preparing them are described in U.S. Pat. Nos. 5,071,919; 5,137,978; 5,137,980; 5,286,823, 5,408,018, 6,562,913, 6,683,138, 7,037,999 and U.S. Publication Nos. 20040176552A1, 20050137363 and 20060079652A1, which are expressly incorporated herein by reference. Such products are commercially available from BASF, under the tradename GLISSOPAL® and from Texas Petrochemicals LP, under the tradename TPC 1105™ and TPC 595™.

In still other embodiments R⁴ hydrocarbyl group in Formula I and/or Formula II can comprise a polyisobutylene substituent derived from a conventional PIB and a high vinylidene PIB with a number average molecular weight as described above.

A conventional PIB can be characterized as having a major amount of a trisubstituted double bond isomer (—C(CH₃)₂C(CH₃)=CHCH₃) and minor amounts of a tetrasubstituted double bond isomer and of an alpha- and/or beta-vinylidene double bond isomer. Conventional PIBs generally can contain a) 45 mole % or greater, 50 mole % or greater, 55 mole % or greater, 45 to 85 mole %, 50 to 75 mole %, or 55 to 70 mole % of trisubstituted double bond isomer, b) 5 to 45 mole %, 10 to 35 mole %, 15 to 30 mole %, or 20 to 25 mole % of tetrasubstituted double bond isomer, c) 30 mole % or less, 25 mole % or less, 1 to 30 mole %, 2 to 30 mole %, or 5 to 25 mole % of alpha- and/or beta-vinylidene double bond isomer, and can have d) a 1.1 to 4, 1.2 to 3.5, or 1.5 to 3 polydispersity defined as the ratio of weight average molecular weight to number average molecular weight. In an embodiment of the invention the conventional PIB has a vinylidene double bond isomer content as described above that comprises the alpha-vinylidene double bond isomer. Conventional PIBs are prepared by polymerizing isobutylene or an isobutylene containing composition, such as a C₄ hydrocarbon stream from a petroleum catalytic cracking unit, with an active acidic polymerization catalyst such as AlCl₃. Conventional PIBs are available commercially under numerous trade names including Parapol® from Exxon and Lubrizol® 3104 from Lubrizol.

A high vinylidene PIB can be characterized as having a major amount of an alpha- and/or beta-vinylidene double bond isomer (respectively —CH2C(CH₃)═CH₂ and/or —CH═C(CH₃)₂) and minor amounts of other isomers including a tetrasubstituted double bond isomer. Because of their high vinylidene double bond isomer content, high vinylidene PIBs are considered to be more reactive and to undergo a higher conversion to derivatives which are better performers in comparison to derivatives from conventional PIBs. High vinylidene PIBs generally can contain a) 70 mole % or greater, 80 mole % or greater, 90 mole % or greater, 70 to 99.9 mole %, 80 to 99.5 mole %, or 85 to 99 mole % of alpha- and/or beta-vinylidene double bond isomer, b) 0.1 to 15 mole %, 0.5 to 12 mole %, or 1 to 10 mole % of tetrasubstituted double bond isomer, and can have c) a 1.0 or 1.1 to 3.5, a 1.2 to 3, or a 1.3 to 2.5 polydispersity. In an embodiment of the invention the high vinylidene PIB can have an alpha-vinylidene double bond isomer content of 75 to 95 mole % or 80 to 90 mole %, and in another embodiment the high vinylidene PIB can have an alpha-vinylidene double bond isomer content of 50 to 70 mole % or 55 to 65 mole %. High vinylidene PIBs are prepared by polymerizing isobutylene or an isobutylene containing composition with a milder acidic polymerization catalyst such as BF₃. High vinylidene PIBs are available commercially from several producers to include BASF and Texas Petroleum Chemicals.

The polyisobutylene substituent derived from a conventional PIB and a high vinylidene PIB can have a) an alpha- and/or beta-vinylidene double bond isomer content of 97 mole % or less, 85 mole % or less, 75 mole % or less, less than 70 mole %, 50 to 95 or 97 mole %, 55 to 80 mole %, 60 to 75 mole %, or 55 to 69 mole %, b) a trisubstituted double bond isomer content of 4 or 5 to 40 mole %, 10 to 30 mole %, or 15 to 25 mole %, c) a tetrasubstituted double bond isomer content of 5 to 20 mole %, 6 to 18 mole %, or 7 to 15 mole %, and can have d) a polydispersity of 1.1 to 3.8, 1.2 to 3.5, or 1.3 to 2.8.

In one embodiment of the invention the PIB can generally have 50 to 95 mole % of alpha- and/or beta-vinylidene double bond isomer and 4 to 40 mole % of trisubstituted double bond isomer, and in other embodiments can have 60 to 75 or 55 to 69 mole % of alpha- and/or beta-vinylidene double bond isomer and 15 to 25 mole % of trisubstituted double bond isomer. In a further embodiment of the invention the PIB of the PIB alkylated hydroxyaromatic compound is derived from a conventional PIB and high vinylidene PIB where the weight ratio of conventional PIB to high vinylidene PIB is respectively 0.1:99.9 to 99.9:0.1, 15:85 to 60:40, or 25:75 to 40:60.

In other embodiments R⁴ may be described as a polyisobutylene group with a molecular weight of 350 to 5000, 500 to 2500 or 750 to 1200. In some embodiments, the groups R¹, R², R³ and R⁶ are each independently either hydrogen or a hydrocarbyl group containing 1 to 6, 1 to 4, or 1 to 3 carbon atoms.

In one embodiment, the fuel detergent additive is represented by Formula II, shown above, wherein: R¹ is independently hydrogen or a hydrocarbyl group containing 1 to 50, 1 to 25, 1 to 10, or 1 to 6 carbon atoms; R² is independently a hydrocarbyl group containing 1 to 10, or 1 to 6 carbon atoms; n is 0 or 1; R³ is independently hydrogen or a hydrocarbyl group containing 1 to 50, 1 to 25, 1 to 10, or 1 to 6 carbon atoms; R⁴ is a hydrocarbyl group containing 1 to 150, 10 to 150, or 50 to 150 carbon atoms; R⁶ is independently hydrogen or a hydrocarbyl group containing 1 to 50 carbon atoms or —(R⁷)ₘ—OR⁸ wherein R7 is a divalent hydrocarbon group containing 1 to 10 carbon atoms, R8 is hydrogen or a hydrocarbyl group containing 1 to 50 carbon atoms and m is 0 or 1; and wherein R¹, R², R³, R⁴, and R⁶ are free of nitrogen. In other embodiments R⁶ is independently hydrogen or a hydrocarbyl group containing 1 to 50, 1 to 25, 1 to 10, or 1 to 6 carbon atoms.

In other embodiments, the present invention is represented by Formula II wherein: R¹ is hydrogen or a hydrocarbyl group containing 1 to 6, 1 to 4, or 1 to 3 carbon atoms; R² is a hydrocarbyl group containing 1 to 6, 1 to 4, or 1 to 3 carbon atoms; R³ is hydrogen or a hydrocarbyl group containing 1 to 6, 1 to 4, or 1 to 3 carbon atoms; R⁴ is a polyisobutylene group having a number average molecular weight of 350 to 5000, or 500 to 2500, 550 to 2000, or 750 to 1100; and R⁶ is hydrogen or a hydrocarbyl group containing 1 to 6, 1 to 4, or 1 to 3 carbon atoms.

In some embodiments the R² group described above in Formula I and/or Formula II is a methylene group. In some of these embodiments the R³ group is a methyl group and the R⁶ group is a hydrogen or a methyl group. In some of these embodiments R⁴ is a polyisobutylene group with a number average molecular weight of 500 to 2500.

In one embodiment the fuel detergent additive is prepared by reacting a hydrocarbyl-substituted hydroxy aromatic compound with an aldehyde, optionally in the presence of a base catalyst. In another embodiment the hydrocarbyl-substituted hydroxy aromatic compound is a hydrocarbyl phenol, a hydrocarbyl cresol, or a mixture thereof.

The hydrocarbyl-substituted hydroxy aromatic compounds suitable for use in the present invention have at least one hydrocarbyl group attached to the ring structure. In some embodiments the compound has only one hydrocarbyl group, however in other embodiments the compound may have 2, 3 4 or five hydrocarbyl groups in addition to the hydroxy group, all attached to the ring structure. The hydrocarbyl group in the para position to the hydroxy group is typically the largest group while the other hydrocarbyl groups tend to be smaller, if they are present at all.

In some embodiments the hydrocarbyl groups that are not in the para position (represented by R², R³, R⁵ and R⁶ in Formula I and Formula II shown above) contain from 1 to 10 carbon atoms, 1 to 6 carbon atoms, or 1 to 4 carbon atoms. In other embodiments these hydrocarbyl groups are each independently hydrogen or methyl groups, such as the group present in cresol.

The hydrocarbyl group located in the para position (represented by R⁴ in Formula I and Formula II shown above) generally contains an average of at least 8, or 30, or 35 up to 350, or to 200, or to 100 carbon atoms. In one embodiment, the hydrocarbyl group is derived from a polyalkene.

Suitable polyalkenes include homopolymers and interpolymers of polymerizable olefin monomers of 2 to 16 or to 6, or to 4 carbon atoms. The olefins may be monoolefins such as ethylene, propylene, 1-butene, isobutylene, and 1-octene; or a polyolefinic monomer, such as diolefinic monomer, such 1,3-butadiene and isoprene. In one embodiment, the interpolymer is a homopolymer. An example of a polymer is a polybutene. In one instance 50% of the polybutene is derived from isobutylene. The polyalkenes are prepared by conventional procedures. In one embodiment of the invention the hydrocarbyl substituent of the hydrocarbyl-substituted hydroxy aromatic compound is derived from a polyisobutylene.

In one embodiment, the hydrocarbyl groups are derived from polyalkenes having a number average molecular weight of least 250, 350, 500, or 750 up to 5000, or to 3000, or to 2000, or to 1500. In some embodiments the polyalkene is polyisobutylene with a molecular weight of 800 to 1200.

The hydroxy aromatic compound from which the hydrocarbyl-substituted hydroxy aromatic compound is derived can comprise phenol, a polyhydroxy benzene such as catechol, an alkyl-substituted phenol such as ortho-cresol, an alkyl-substituted polyhydroxy benzene such as 3-methylcatechol, or mixtures thereof. The hydrocarbyl-substituted hydroxy aromatic compound can be prepared by well known alkylation methods generally involving alkylation of the hydroxy aromatic compound with a polyolefin in the presence of acidic catalyst. The acidic catalyst can include for example mineral acids such as a sulfuric acid acidified clay, Lewis acid catalysts such as a complex of boron trifluoride with diethyl ether or with phenol, and acidic ion exchange resins such as the Amberlyst® series of strongly acidic macroreticular resins available from Rohm and Haas. In an embodiment of the invention phenol is alkylated with a conventional polyisobutylene, a highly reactive polyisobutylene or a mixture of conventional and highly reactive polyisobutylenes in the presence of a solvent or diluent and a BF₃ catalyst between 0 and 50° C. as described in U.S. Pat. No. 5,876,468.

While the term “phenol” is used herein, it is to be understood that this term is not intended to limit the aromatic group of the phenol to benzene. Accordingly, it is to be understood that the aromatic group represented in this specification may be mononuclear or polynuclear.

The aldehydes suitable for use in the present invention are hydrocarbon-based aldehydes, preferably lower aliphatic aldehydes. Suitable aldehydes include formaldehyde, benzaldehyde, acetaldehyde, the butyraldehydes, hydroxybutyraldehydes and heptanals, as well as aldehyde precursors which react as aldehydes under the conditions of the reaction such as paraformaldehyde, paraldehyde, and formalin. In some embodiments the aldehyde is Formaldehyde and/or its precursors and reaction synthons (e.g., paraformaldehyde, trioxane). Mixtures of aldehydes may be used to prepare the additive of the present invention.

The catalyst is not overly limited and may include an esterification catalyst such as toluenesulfonic acid, sulfuric acid, aluminum chloride, boron trifluoride-triethylamine, methanesulfonic acid, hydrochloric acid, ammonium sulfate, phosphoric acid, sodium methoxide and the like. These conditions and variations thereof are well known in the art. In one embodiment the additive of the present invention is prepared in the presence of sodium methoxide.

The fuel detergents of the present invention can be solids, semi-solids, or liquids (oils) depending on the particular alcohol(s) and/or amine(s) used in preparing them. For use as additives in oleaginous compositions including lubricating and fuel compositions the fuel detergents are advantageously soluble and/or stably dispersible in such oleaginous compositions. Thus, for example, compositions intended for use in fuels are typically fuel-soluble and/or stably dispersible in a fuel in which they are to be used. The term “fuel-soluble” as used in this specification and appended claims does not necessarily mean that all the compositions in question are miscible or soluble in all proportions in all fuels. Rather, it is intended to mean that the composition is soluble in a fuel (hydrocarbon, non-hydrocarbon, mixtures, etc) in which it is intended to function to an extent which permits the solution to exhibit one or more of the desired properties. Similarly, it is not necessary that such “solutions” be true solutions in the strict physical or chemical sense. They may instead be micro-emulsions or colloidal dispersions which, for the purpose of this invention, exhibit properties sufficiently close to those of true solutions to be, for practical purposes, interchangeable with them within the context of this invention.

As previously indicated, the nitrogen-free fuel detergent additives of this invention are useful as additives for fuels, in which they may function as detergents. The fuel detergents of the present invention can be present in fuel compositions at 1 to 10,000 ppm (where ppm is calculated on a weight:weight basis). In additional embodiments, the fuel detergent is present in fuel compositions in ranges with lower limits of 1, 5, 10, 20, 50, 100, 150 and 200 ppm and upper limits of 10,000, 5,000, 2,500, 1,000, and 500 where any upper limit may be combined with any lower limit to provide a range for the fuel detergent present in the fuel compositions. In one embodiment the fuel detergent is present at 10 to 2500 ppm, and in another embodiment from 20-500 ppm.

### The Fuel Additive Compositions

The fuel additive composition of the present invention comprises the nitrogen-free fuel detergent additive described herein and further comprises a solvent and/or one or more additional performance additives. These additive compositions, also known as concentrates, may be used to prepare fuel compositions by adding the additive composition to a fuel.

The solvents suitable for use in the present invention include hydrocarbon solvents that provide for the additive composition's compatibility and/or homogeneity and to facilitate their handling and transfer and may include a fuel as described below. The solvent can be an aliphatic hydrocarbon, an aromatic hydrocarbon, an oxygen-containing composition, or a mixture thereof. In some embodiments the flash point of the solvent is generally about 25° C. or higher. In some embodiments the hydrocarbon solvent is an aromatic naphtha having a flash point above 62° C. or an aromatic naphtha having a flash point of 40° C. or a kerosene with a 16% aromatic content having a flash point above 62° C.

Aliphatic hydrocarbons include various naphtha and kerosene boiling point fractions that have a majority of aliphatic components. Aromatic hydrocarbons include benzene, toluene, xylenes and various naphtha and kerosene boiling point fractions that have a majority of aromatic components. Alcohols are usually aliphatic alcohols having about 2 to 10 carbon atoms and include ethanol, 1-propanol, isopropyl alcohol, 1-butanol, isobutyl alcohol, amyl alcohol, and 2-methyl-l-butanol.

The oxygen containing composition can include an alcohol, a ketone, an ester of a carboxylic acid, a glycol and/or a polyglycol, or a mixture thereof. The solvent in an embodiment of the invention will be substantially free of to free of sulphur having a sulphur content in several instances that is below 50 ppm, 25 ppm, below 18 ppm, below 10 ppm, below 8 ppm, below 4 ppm, or below 2 ppm. The solvent can be present in the additive concentrate composition at 0 to 99 percent by weight, and in other instances at 3 to 80 percent by weight, or 10 to 70 percent by weight. The fuel addditive of the present invention and the additional performance additives taken separately or in combination can be present in the additive concentrate composition at 0.01 to 100 percent by weight, and in other instances can be present at 0.01 to 95 percent by weight, at 0.01 to 90 percent by weight, or at 0.1 to 80 percent by weight.

As allowed for by the ranges above, in one embodiment, the additive concentrate may comprise the fuel detergent of the present invention and be substantially free of any additional solvent. In these embodiments the additive concentrate containing the fuel detergent of the present invention is neat, in that it does not contain any additional solvent added to improve the material handling characteristics of the concentrate, such as its viscosity. However, in other embodiments, the additive concentrate containing the additive of the present invention does contain some solvent.

In an embodiment of the invention the additive concentrate composition, or a fuel composition containing the fuel detergent of the present invention, may be prepared by admixing or mixing the components of the composition at ambient to elevated temperatures usually up to 60° C. until the composition is homogeneous.

In some embodiments the fuel additive composition is substantially nitrogen free or nitrogen free. In other embodiments the fuel additive composition comprises the nitrogen free fuel additive described above but also comprises additional additive which may not be nitrogen free.

The additional performance additives which may be included in the additive compositions of the present invention are described below.

### The Fuel

The fuel composition of the present invention comprises the fuel detergent described above and a liquid fuel, and is useful in fueling an internal combustion engine. A fuel may also be a component of the additive compositions described above.

Fuels suitable for use in the present invention are not overly limited. Generally, suitable fuels are normally liquid at ambient conditions e.g., room temperature (20 to 30° C.). The liquid fuel can be a hydrocarbon fuel, a non-hydrocarbon fuel, or a mixture thereof.

The hydrocarbon fuel can be a petroleum distillate, including a gasoline as defined by ASTM specification D4814, or a diesel fuel, as defined by ASTM specification D975. In one embodiment the liquid fuel is a gasoline, and in another embodiment the liquid fuel is a non-leaded gasoline. In another embodiment the liquid fuel is a diesel fuel. The hydrocarbon fuel can be a hydrocarbon prepared by a gas to liquid process to include for example hydrocarbons prepared by a process such as the Fischer-Tropsch process.

The non-hydrocarbon fuel can be an oxygen containing composition, often referred to as an oxygenate, which includes an alcohol, an ether, a ketone, an ester of a carboxylic acid, a nitroalkane, or a mixture thereof. The non-hydrocarbon fuel can include for example methanol, ethanol, butanol, methyl t-butyl ether, methyl ethyl ketone, transesterified oils and/or fats from plants and animals such as rapeseed methyl ester and soybean methyl ester, and nitromethane.

Mixtures of hydrocarbon and non-hydrocarbon fuels can include, for example, gasoline and methanol and/or ethanol, diesel fuel and ethanol, and diesel fuel and a transesterified plant oil such as rapeseed methyl ester and other bio-derived fuels. In one embodiment the liquid fuel is an emulsion of water in a hydrocarbon fuel, a non-hydrocarbon fuel, or a mixture thereof. In several embodiments of this invention the liquid fuel can have a sulphur content on a weight basis that is 5000 ppm or less, 1000 ppm or less, 300 ppm or less, 200 ppm or less, 30 ppm or less, or 10 ppm or less.

In some embodiments the fuel composition is substantially nitrogen free or nitrogen free. In other embodiments the fuel composition comprises the nitrogen free fuel additive described above but also comprises additional additive which may not be nitrogen free.

The liquid fuel of the invention is present in a fuel composition in a major amount that is generally greater than 95% by weight, and in other embodiments is present at greater than 97% by weight, greater than 99.5% by weight, or greater than 99.9% by weight.

### Additional Performance Additives

The additive compositions and fuel compositions of the present invention can further comprise one or more additional performance additives. Additional performance additives can be added to a fuel composition depending on several factors to include the type of internal combustion engine and the type of fuel being used in that engine, the quality of the fuel, and the service conditions under which the engine is being operated. In some embodiments the additional performance additives added are free of nitrogen. In other embodiments, the additional performance additives may contain nitrogen.

The additional performance additives can include: an antioxidant such as a hindered phenol or derivative thereof and/or a diarylamine or derivative thereof; a corrosion inhibitor such as an alkenylsuccinic acid; and/or a detergent/dispersant additive, other than the fuel detergent of the present invention, such as a polyetheramine or nitrogen containing detergent, including but not limited to PIB amine dispersants, quaternary salt dispersants, and succinimide dispersants.

The additional performance additives may also include: a cold flow improver such as an esterified copolymer of maleic anhydride and styrene and/or a copolymer of ethylene and vinyl acetate; a foam inhibitor such as a silicone fluid; a demulsifier such as a polyalkoxylated alcohol; a lubricity agent such as a fatty carboxylic acid; a metal deactivator such as an aromatic triazole or derivative thereof, including but not limited to benzotriazole; and/or a valve seat recession additive such as an alkali metal sulfosuccinate salt. The additional additives may also include a biocide; an antistatic agent, a deicer, a fluidizer such as a mineral oil and/or a poly(alpha-olefin) and/or a polyether, and a combustion improver such as an octane or cetane improver.

The additional performance additives which may be present in the fuel additive compositions and fuel compositions of the present invention also include di-ester, di-amide, ester-amide, and ester-imide friction modifiers prepared by reacting a dicarboxylic acid (such as tartaric acid) and/or a tricarboxyli acid (such as citric acid), with an amine and/or alcohol, optionally in the presence of a known esterification catalyst. These friction modifiers, often derived from tartaric acid, citric acid, or derivates thereof, may be derived from amines and/or alcohols that are branched so that the friction modifier itself has significant amounts of branched hydrocarbyl groups present within it structure. Examples of a suitable branched alcohols used to prepare these friction modifiers include 2-ethylhexanol, isotridecanol, Guerbet alcohols, or mixtures thereof

The additional performance additives can each be added directly to the additive and/or the fuel compositions of the present invention, but they are generally mixed with the nitrogen-free fuel detergent additive to form an additive composition, or concentrate, which is then mixed with fuel to result in a fuel composition. The additive concentrate compositions are described in more detail above.

### INDUSTRIAL APPLICATION

In one embodiment the invention is useful for a liquid fuel and/or for an internal combustion engine, including either compression ignition engines or spark ignited engines. The internal combustion engine includes 2-stroke or 4-stroke engines fuelled with gasoline, diesel, a natural gas, a mixed gasoline/alcohol or any of the fuels described in the sections above. The compression ignition engines include both light duty and heavy duty diesel engines. The spark ignited engines include direct injection gasoline engines.

In other embodiments the invention is useful in additive compositions in that the fuel detergent described above provides improved engine deposit control, allowing for improved engine performance, including but not limited to reductions in deposit-caused engine power losses, reduction in deposit-caused fuel economy losses and decreases in deposit-caused engine emissions.

In still other embodiments the additive compositions of the present invention may be used in a lubricating composition such that the additives are present in the lubricating system of the engine. The additives may also enter the combustion chamber of the engine during operation of the engine by the transfer of small amounts of the additive containing lubricating composition to the combustion chamber due to a phenomenon referred to as “blow by” where the lubricating composition, and in this case the additive composition, pass around the piston heads inside the cylinder, moving from the lubricating system of the engine into the combustion chamber.

As used herein, there “nitrogen free” is used in its ordinary sense and means that the fuel detergent additive of the present invention contains no nitrogen atoms. The invention is not limited to nitrogen-free compositions, as other nitrogen-containing substances may be added to compositions that include the nitrogen-free fuel detergent described herein. However, in some embodiments, the nitrogen content of the additive compositions and/or the fuel compositions of the present invention are less than 100 ppm, less than 50 ppm, less than 35 ppm or less than 10 ppm (where ppm is calculated on a weight: weight basis). In still other embodiments, the additive and/or fuel compositions of the present invention are free of nitrogen.

As used herein, the term “hydrocarbyl substituent” or “hydrocarbyl group” is used in its ordinary sense, which is well-known to those skilled in the art. Specifically, it refers to a group having a carbon atom directly attached to the remainder of the molecule and having predominantly hydrocarbon character. Examples of hydrocarbyl groups include: hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form a ring); substituted hydrocarbon substituents, that is, substituents containing non-hydrocarbon groups which, in the context of this invention, do not alter the predominantly hydrocarbon nature of the substituent (e.g., halo (especially chloro and fluoro), hydroxy, alkoxy, mercapto, alkylmercapto, nitro, nitroso, and sulfoxy); hetero substituents, that is, substituents which, while having a predominantly hydrocarbon character, in the context of this invention, contain other than carbon in a ring or chain otherwise composed of carbon atoms. Heteroatoms include sulfur, oxygen, nitrogen, and encompass substituents as pyridyl, furyl, thienyl and imidazolyl. In general, no more than two, preferably no more than one, non-hydrocarbon substituent will be present for every ten carbon atoms in the hydrocarbyl group; typically, there will be no non-hydrocarbon substituents in the hydrocarbyl group.

It is known that some of the materials described above may interact in the final formulation, so that the components of the final formulation may be different from those that are initially added. For instance, metal ions (of, e.g., a detergent) can migrate to other acidic or anionic sites of other molecules. The products formed thereby, including the products formed upon employing the composition of the present invention in its intended use, may not be susceptible of easy description. Nevertheless, all such modifications and reaction products are included within the scope of the present invention; the present invention encompasses the composition prepared by admixing the components described above.

### EXAMPLES

The invention will be further illustrated by the following examples, which sets forth particularly advantageous embodiments. While the examples are provided to illustrate the present invention, they are not intended to limit it.

Samples of fuel compositions, including several which contain nitrogen-free fuel detergent additives of the present invention, are tested to evaluate their ability to control engine deposits, specifically intake value deposits (IVD).

### Comparative Example 1

A commercially available nitrogen-containing additive is prepared by reacting 100 pbw mid vinylidene 1000 Mn Polyisobutylene (TPC 1105™ available from the Texas Petrochemicals LP) with with 8 pbw of an aldehyde and 11 pbw of a polyalkylamine in a reaction vessel which also contains a viscosity-controlling amount of organic solvent. The reaction is carried out below 85° C. and aqueous distillate was removed from the system. The reaction product is a nitrogen-containing additive.

### Comparative Example 2

PiB phenol, derived from mid vinylidene 1000 Mn Polyisobutylene (TPC 1105™ available from the Texas Petrochemicals LP) and phenol, is well known in the art. It is used herein as a comparative example in the testing below. This material, as the results below show, does not perform well as a fuel additive. This poor performance is expected given the lack of nitrogen, or more specifically, the lack of a polar nitrogen group, in the additive. This same result would be expected for the additives of the present invention as they too lack a polar nitrogen group.

### Example 1

A nitrogen-free fuel additive is prepared by mixing 1000 grams of mid vinylidene 1000 Mn Polyisobutylene (TPC 1105™ available from the Texas Petrochemicals LP) with 217 grams of toluene. The mixture is then charged to a reaction vessel. To the reaction vessel 281.2 grams of ortho cresol and 122 grams of toluene are charged, and the system is stirred for 15 minutes under a nitrogen blanket. Over 3 hours, 19.9 grams of boron-trifluoride etherate is added to the reaction vessel in a dropwise manner while the mixture in the reaction vessel is stirred and kept below 25° C. After the addition is complete, the mixture in the reaction vessel is stirred for 3 hours at ambient temperature. Then 41.5 grams of lime and 41.5 grams of Fax-5 filter aid is then added to the reaction vessel, which is then allowed to stir overnight. The reaction mixture is then filtered using a sinter glass filter funnel and a Fax-5 filtering pad. After filtration the filtrate is charged to a vessel and vacuum stripped at 0.8 bar and 110° C. for 60 minutes and then is further vacuum stripped at 0.8 bar and 205° C. for 270 minutes. The material remaining in the vessel is cooled to 40° C. and collected. The collected material contains a nitrogen free additive.

### Example 2

A nitrogen-free fuel additive is prepared by mixing 165 grams of PiB cresol, derived from mid vinylidene 1000 Mn Polyisobutylene (TPC 1105™ available from the Texas Petrochemicals LP) and toluene (as described in the first part of Example 1 above), and 47.8 grams of methanol and 6.8 grams of paraformaldehyde in a reaction vessel. The reaction mixture is then stirred and heated to 55° C. under a nitrogen blanket. Then 32.4 grams of a mixture of sodium methoxide at 25% actives in methanol is added to the reaction vessel subsurface over 70 minutes. After the charge is complete, the reaction mixture is heated to 60° C. and then held at temperature for 150 minutes. The reaction mixture is then cooled back to ambient temperature and then transferred to a rotary evaporator. The material is vacuum stripped at 0.7 bar and 105° C. to remove the methanol. The product is then cooled and collected. The collected material contains a nitrogen free additive.

### Example 3

A nitrogen-free fuel additive is prepared by mixing 250 grams of PiB cresol, derived from mid vinylidene 1000 Mn Polyisobutylene (TPC 1105™ available from the Texas Petrochemicals LP) and toluene (as described in the first part of Example 1 above), and 73.0 grams of methanol and 10.2 grams of paraformaldehyde in a reaction vessel. The reaction mixture is then stirred and heated to 55° C. under a nitrogen blanket. Then 49.7 grams of a mixture of sodium methoxide at 25% actives in methanol is added to the reaction vessel subsurface over 70 minutes. After the charge is complete, the reaction mixture is heated to 60° C. and then held at temperature for 150 minutes. The reaction mixture is then cooled back to ambient temperature and then transferred to a rotary evaporator. The material is vacuum stripped at 0.7 bar and 70° C. to remove the methanol. The product is then cooled and collected. The collected material contains a nitrogen free additive.

The examples described above are tested in the M111E engine test (CEC SG-F-0202), which measures Inlet Valve Deposits (IVD). The lower the IVD deposit measured in the test, the more efficient the detergent additive. All examples were added to a standard gasoline test fuel at the treat rates described below.

TABLE 1
M111E Engine test data

| | | Treat rate | Treat rate | Measured |
|---|---|---|---|---|
| Test | Additive | (PIB actives, | (detergent actives*, | Inlet Valve |
| Run | Present | ppm m/m) | ppm m/m) | Deposit |
| A | Comparative | 105 | 100 | 38 mg |
| | Example 1 | | | |
| B | Comparative | 140 | 0 | 115 mg |
| | Example 2 | | | |
| C | Example 2 | 140 | 83 | 20 mg |
| D | Example 3 | 140 | 83 | 18 mg |

*detergent actives defined as derivatized PIB Phenol

The results show that Examples 2 and 3, which contain nitrogen free additives of the present invention, exhibit a greater detergent efficiency than the typical polyalkylphenol amine detergent of Comparative Example 1.

The table also shows that the non-nitrogen containing PiB phenol, of Comparative Example 2, gives the poor performance expected from such an additive. In contrast the nitrogen-free additives of the present invention provide unexpected good detergency, despite lacking a nitrogen group

Each of the documents referred to above is incorporated herein by reference. Except in the Examples, or where otherwise explicitly indicated, all numerical quantities in this description specifying amounts of materials, reaction conditions, molecular weights, number of carbon atoms, and the like, are to be understood as modified by the word “about.” Unless otherwise indicated, all percent values shown herein are weight percents. Unless otherwise indicated, each chemical or composition referred to herein should be interpreted as being a commercial grade material which may contain the isomers, by-products, derivatives, and other such materials which are normally understood to be present in the commercial grade. However, the amount of each chemical component is presented exclusive of any solvent or diluent, which may be customarily present in the commercial material, unless otherwise indicated. It is to be understood that the upper and lower amount, range, and ratio limits set forth herein may be independently combined. Similarly, the ranges and amounts for each element of the invention can be used together with ranges or amounts for any of the other elements. As used herein, the expression “consisting essentially of” permits the inclusion of substances that do not materially affect the basic and novel characteristics of the composition under consideration.

In addition, all the embodiments described above have been contemplated as to their use, both alone and in combination, with all of the other embodiments described above, and these combinations are considered to be part of the present invention.

What we claim:

1. A composition comprising a nitrogen-free detergent fuel additive represented by Formula I; 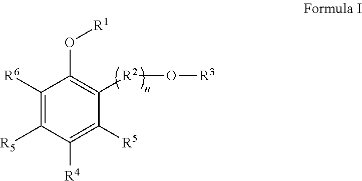
wherein:
R¹ is independently hydrogen or a hydrocarbyl group containing 1 to 50 carbon atoms;
R² is independently a hydrocarbyl group containing 1 to 10 carbon atoms;
n is 0 or 1;
R³ is independently hydrogen or a hydrocarbyl group containing 1 to 50 carbon atoms;
R⁴ is a hydrocarbyl group containing 1 to 150 carbon atoms;
each R⁵ is independently hydrogen or a hydroxyl group;
R⁶ is independently hydrogen or a hydrocarbyl group containing 1 to 50 carbon atoms or —(R⁷)ₘ—OR⁸ wherein R7 is a hydrocarbyl group containing 1 to 10 carbon atoms, R⁸ is hydrogen or a hydrocarbyl group containing 1 to 50 carbon atoms and m is 0 or 1; and
wherein R¹, R², R³, R⁴, R⁵, R⁶, R⁷ and R⁸ are free of nitrogen.

2. The composition of claim 1 wherein R⁴ is derived from polyisobutylene having a number average molecular weight of 350 to 5000.

3. The composition of claim 1 wherein the additive is represented by Formula II: 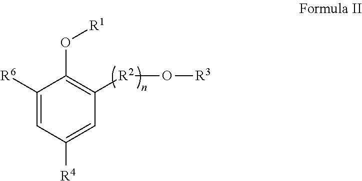
wherein:
R¹ is hydrogen or a hydrocarbyl group containing 1 to 6 carbon atoms;
R² is a methylene group and n is 0 or 1;
R³ is hydrogen or a hydrocarbyl group containing 1 to 6 carbon atoms;
R⁴ is derived from polyisobutylene having a number average molecular weight of 500 to 2500;
each R5 is independently hydrogen or a hydroxyl group; and
R⁶ is independently hydrogen or a hydrocarbyl group containing 1 to 50 carbon atoms or —(R⁷)ₘ—OR⁸ wherein R7 is a hydrocarbyl group containing 1 to 10 carbon atoms, R⁸ is hydrogen or a hydrocarbyl group containing 1 to 50 carbon atoms and m is 0 or 1.

4. The composition of claim 3 wherein the additive comprises a mixture of two or more additives selected from the group consisting of:
a) the additive represented by Formula II wherein R³ is hydrogen and R⁶ is hydrogen;
b) the additive represented by Formula II wherein R³ is a hydrocarbyl group containing 1 to 6 carbon atoms and R⁶ is hydrogen;
c) the additive represented by Formula II wherein R³ is hydrogen and R⁶ is a hydrocarbyl group containing 1 to 6 carbon atoms;
d) the additive represented by Formula II wherein R³ is a hydrocarbyl group containing 1 to 6 carbon atoms and R⁶ is a hydrocarbyl group containing 1 to 6 carbon atoms.

5. The composition of claim 3 wherein:
R¹ is hydrogen;
R² is independently a methylene and n is 0 or 1;
R³ is independently hydrogen or a methyl group
R⁶ is independently hydrogen, a hydroxyl group, or a methyl group.

6. A fuel additive composition comprising:
(a) the nitrogen-free detergent additive of claim 1;
(b) an optional solvent; and
(c) one or more optional additional performance additives;

7. The fuel additive composition of claim 6 wherein component (c) comprises an antioxidant, a corrosion inhibitor, another detergent/dispersant additive, a cold flow, a foam inhibitor, a demulsifier, a lubricity agent, a metal deactivator, a valve seat recession additive, a biocide, an antistatic agent, a deicer, a fluidizer, a combustion improver, a friction modifier, or some combination thereof.

8. A fuel composition comprising:
(a) the nitrogen-free detergent additive of claim 1;
(b) a fuel; and
(c) one or more optional additional performance additives;

9. The fuel composition of claim 8 wherein component (a) is present in an amount of about 10 ppm to 2500 ppm

10. A method of operating an internal combustion engine, comprising supplying to said engine a fuel composition comprising:
(a) a nitrogen-free detergent additive represented by Formula I; and
(b) a fuel; 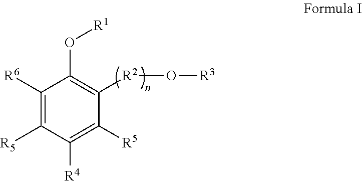
wherein:
R¹ is independently hydrogen or a hydrocarbyl group containing 1 to 50 carbon atoms;
R² is independently a hydrocarbyl group containing 1 to 10 carbon atoms;
n is 0 or 1;
R³ is independently hydrogen or a hydrocarbyl group containing 1 to 50 carbon atoms;
R⁴ is a hydrocarbyl group containing 1 to 150 carbon atoms;
each R⁵ is independently hydrogen or a hydroxyl group;
R⁶ is independently hydrogen or a hydrocarbyl group containing 1 to 50 carbon atoms or —(R⁷)ₘ—OR⁸ wherein R7 is a hydrocarbyl group containing 1 to 10 carbon atoms, R8 is hydrogen or a hydrocarbyl group containing 1 to 50 carbon atoms and m is 0 or 1; and
wherein R¹, R², R³, R⁴, R⁵, R⁶, R⁷ and R⁸ are free of nitrogen.
